(19) United States
(12) Patent Application Publication
(10) Pub. No.: US 2016/0354584 A1
(43) Pub. Date: Dec. 8, 2016

(54) TUBULAR COMPONENT DESIGN AND METHOD OF MANUFACTURE

(71) Applicant: BOSTON SCIENTIFIC SCIMED, INC., MAPLE GROVE, MN (US)

(72) Inventors: BRIAN J. HANSON, SHOREVIEW, MN (US); STEVEN KOCH, ZIMMERMAN, MN (US)

(73) Assignee: BOSTON SCIENTIFIC SCIMED, INC., MAPLE GROVE, MN (US)

(21) Appl. No.: 15/175,774

(22) Filed: Jun. 7, 2016

Related U.S. Application Data
(60) Provisional application No. 62/172,766, filed on Jun. 8, 2015.

(51) Int. Cl. A61M 25/09; A61M 25/00

(52) U.S. Cl. CPC A61M 25/09; A61M 25/0013; A61B 2017/00526

(57) ABSTRACT

A method of manufacturing a medical device is disclosed. The method comprises laser cutting a tubular member. The tubular member may have an inner surface, an outer surface and a tubular wall defining a thickness extending therebetween. The method may also include laser cutting the member. Laser cutting may include removing a portion of the thickness of the tubular wall at one or more discrete locations along the tubular wall. The method may also include chemically etching the one or more discrete locations to form a slot within the tubular wall at the one or more discrete locations along the tubular member.

### CROSS-REFERENCE TO RELATED APPLICATIONS

This application claims priority under 35 U.S.C. §119(e) to U.S. Provisional Application No. 62/172,766, filed Jun. 8, 2015, the entire disclosure of which is herein incorporated by reference.

### FIELD OF THE INVENTION

The present invention pertains to medical devices, and methods for manufacturing medical devices. More particularly, the present invention pertains to elongated intracorporeal medical devices including a slotted tubular member.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example method for manufacturing a medical device is disclosed. The method comprises:

laser cutting a tubular member, the tubular member having an inner surface, an outer surface and a tubular wall defining a thickness extending therebetween, wherein laser cutting the member includes removing a portion of the thickness of the tubular wall at one or more discrete locations along the tubular wall; and

chemically etching the one or more discrete locations to form a slot within the tubular wall at the one or more discrete locations along the tubular member.

Alternatively or additionally to any of the embodiments above, laser cutting includes laser cutting with a femtosecond laser.

Alternatively or additionally to any of the embodiments above, forming a slot within the tubular wall includes removing the tubular wall from the outside surface to the inner surface of the tubular member.

Alternatively or additionally to any of the embodiments above, forming a slot within the tubular wall includes removing a portion of the tubular wall from the outside surface to a location between the outside surface and the inner surface of the tubular member.

Alternatively or additionally to any of the embodiments above, laser cutting the tubular member includes ablating a portion of the tubular wall.

Alternatively or additionally to any of the embodiments above, laser cutting to remove a portion of the thickness of the tubular wall includes removing at least 80% of the thickness of the tubular wall.

Alternatively or additionally to any of the embodiments above, laser cutting creates a first wall portion and a second wall portion, and wherein the first wall portion is longitudinally aligned with the second wall portion and wherein a connecting portion extends between the first wall portion and the second wall portion.

Alternatively or additionally to any of the embodiments above, the connecting portion is continuous.

Alternatively or additionally to any of the embodiments above, the connecting portion is discontinuous.

Alternatively or additionally to any of the embodiments above, further comprising performing laser cutting prior to chemical etching.

Another method of manufacturing a medical device comprises:

laser cutting a tubular member, the tubular member having an inner diameter, an outer diameter and a tubular wall defining a thickness, wherein laser cutting the member includes removing a portion of the thickness of the tubular wall to form one or more cavities in the tubular wall; and

chemically etching the tubular member to form a slot within the tubular wall at the one or more cavities along the tubular member, and wherein chemically etching increases the inner diameter of the tubular member.

Alternatively or additionally to any of the embodiments above, laser cutting includes using a femtosecond laser.

Alternatively or additionally to any of the embodiments above, removing a portion of the thickness of the tubular wall includes removing at least 80% of the tubular wall.

Alternatively or additionally to any of the embodiments above, laser cutting includes ablating a portion of the tubular wall.

Alternatively or additionally to any of the embodiments above, chemically etching the tubular member includes bathing the tubular member in an acid bath while rotating the tubular member, translating the tubular member, or both.

An example medical device is disclosed. The example medical device comprises:

an elongate shaft including a tubular member, the tubular member having an inner surface, an outer surface, a tubular wall extending between the outer surface and the inner surface and a plurality of slots extending from the outer surface to the inner surface; and

wherein the plurality of slots are created by laser cutting one or more cavities in the tubular wall at one or more discrete locations along the outer surface of the tubular member and chemically etching the one or more cavities.

Alternatively or additionally to any of the embodiments above, laser cutting includes using a femtosecond laser.

Alternatively or additionally to any of the embodiments above, laser cutting includes ablation a portion of the tubular wall.

Alternatively or additionally to any of the embodiments above, creating the one or more cavities includes removing a portion of the tubular wall, and wherein chemically etching includes removing the remaining portion of the tubular wall.

Alternatively or additionally to any of the embodiments above, removing a portion of the tubular wall includes removing at least 80% of a thickness of the tubular wall.

### BRIEF DESCRIPTION OF THE DRAWINGS

The invention may be more completely understood in consideration of the following detailed description of various embodiments of the invention in connection with the accompanying drawings, in which:

FIG. 1 is a plan view of an example medical device disposed in a blood vessel;

FIG. 2 is a side view of an example tubular member for use in a medical device;

FIG. 3A is a side view of another example tubular member for use in a medical device prior to laser processing;

FIG. 3B is a side view of another example tubular member after laser processing;

FIG. 3C is a detailed view of the example tubular member illustrated in FIG. 3B;

FIG. 4A is a perspective view of another example tubular member after laser processing and prior to chemical treatment;

FIG. 4B is a cross-sectional view of the example tubular member of FIG. 4A;

FIG. 4C is a cross-sectional view of the example medical device of FIG. 4B after chemical processing;

FIG. 5 is a plan view of another example medical device.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

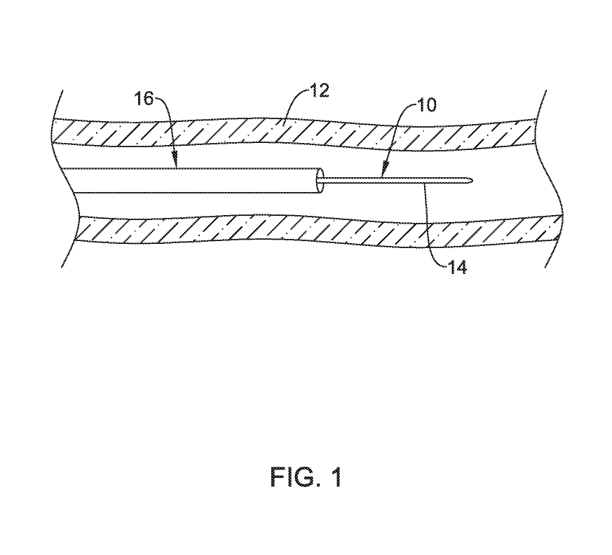

FIG. 1 is a plan view of an example medical device 10, for example a guidewire, disposed in a blood vessel 12. Guidewire 10 may include a distal section 14 is that may be generally configured for use within the anatomy of a patient. Guidewire 10 may be used for intravascular procedures. For example, guidewire 10 may be used in conjunction with another medical device 16, which may take the form of a catheter, to treat and/or diagnose a medical condition. Of course, numerous other uses are known amongst clinicians for guidewires, catheters, and other similarly configured medical devices.

Although medical device 10 is depicted in several of the drawings as a guidewire, it is not intended to be limited to just being a guidewire. Indeed, medical device 10 may take the form of any suitable guiding, diagnosing, or treating device (including catheters, stents, endoscopic instruments and/or endoscopes, laparoscopic instruments, stent delivery systems, embolic filter systems, urology stone retrieval systems, embolic coil delivery systems, atherectomy shafts, thermoctomy shafts, pacing leads, neuromodulation contacts, neuromodulation electrodes, cardiac rhythm management leads and/or contacts, etc., and the like) and it may be suitable for use at essentially any location and/or body lumen within a patient. For example, medical device/guidewire 10 may be suitable for use in neurological interventions, coronary interventions, peripheral interventions, etc. As such, guidewire 10 may be appropriately sized for a given intervention. For example, guidewire 10 may have an outside diameter of about 0.001 to 0.050 inches or about 0.0015 to 0.025 inches. Further, guidewire 10 may have an inner lumen diameter of about 0.001 to 0.050 inches or about 0.005 to 0.025 inches. These dimensions, of course, may vary depending on, for example, the type of device (e.g., catheter, guidewire, etc.), the anatomy of the patient, and/or the goal of the intervention.

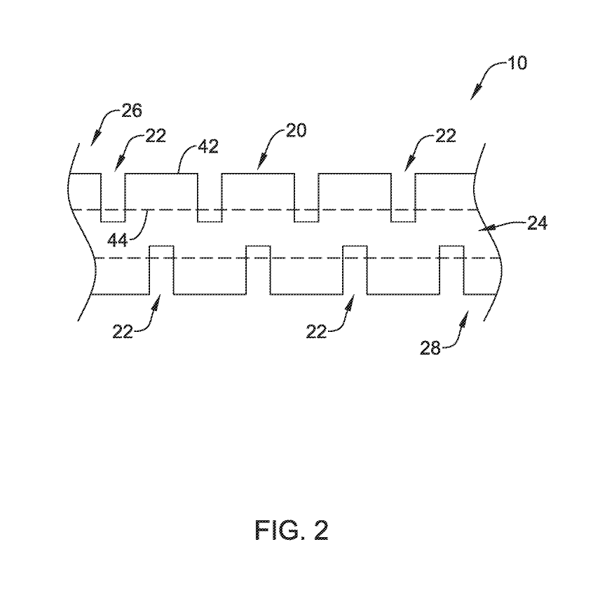

FIG. 2 is a side view of example guidewire 10. Here it can be seen that guidewire 10 may include a tubular member 20 having a plurality of cuts, apertures, and/or slots 22 formed therein. Various embodiments of arrangements and configurations of slots 22 are contemplated. In some embodiments, at least some, if not all of slots 22 are disposed at the same or a similar angle with respect to the longitudinal axis of the tubular member 20. As shown, slots 22 can be disposed at an angle that is perpendicular, or substantially perpendicular, and/or can be characterized as being disposed in a plane that is normal to the longitudinal axis of tubular member 20. However, in other embodiments, slots 22 can be disposed at an angle that is not is perpendicular, and/or can be characterized as being disposed in a plane that is not normal to the longitudinal axis of tubular member 20. Additionally, a group of one or more slots 22 may be disposed at different angles relative to another group of one or more slots 22.

Slots 22 may be provided to enhance the flexibility of tubular member 20 while still allowing for suitable torque transmission characteristics. Slots 22 may be formed such that one or more rings and/or turns interconnected by one or more segments and/or beams are formed in tubular member 20, and such rings and beams may include portions of tubular member 20 that remain after slots 22 are formed in the body of tubular member 20. Such an interconnected ring structure may act to maintain a relatively high degree of torsional stiffness, while maintaining a desired level of lateral flexibility. In some embodiments, some adjacent slots 22 can be formed such that they include portions that overlap with each other about the circumference of tubular member 20. In other embodiments, some adjacent slots 22 can be disposed such that they do not necessarily overlap with each other, but are disposed in a pattern that provides the desired degree of lateral flexibility.

Additionally, slots 22 can be arranged along the length of, or about the circumference of, tubular member 20 to achieve desired properties. For example, adjacent slots 22, can be arranged in a symmetrical pattern, such as being disposed essentially equally on opposite sides about the circumference of tubular member 20, or can be rotated by an angle relative to each other about the axis of tubular member 20. Additionally, adjacent slots 22, may be equally spaced along the length of tubular member 20, or can be arranged in an increasing or decreasing density pattern, or can be arranged in a non-symmetric or irregular pattern. This may include slots 22 that form or otherwise follow a helical pattern about tubular member 20. Other characteristics, such as slot size, slot shape and/or slot angle with respect to the longitudinal axis of tubular member 20, can also be varied along the length of tubular member 20 in order to vary the flexibility or other properties. In other embodiments, moreover, it is contemplated that the portions of the tubular member, such as a proximal section 26, or a distal section 28, or the entire tubular member 20, may not include any such slots 22.

For the purposes of this disclosure, slots 22 may be understood to be cuts or openings that extend through the wall of tubular member 20. For example, FIG. 2 shows slots 22 extending through the wall of tubular member 20 and into inner lumen 24 of tubular member 20. In some instances, forming slots in a tubular member may result in dross, debris, residue, particulate, nano-particulate or the like being deposited and/or accumulating on the inner lumen 24 of tubular member 20. Further, in some instances additional processing may be required to remove the accumulated dross, debris, residue, particulate, etc. Therefore, it may be desirable to process the slots such that they extend through the wall of the tubular member, yet prevent debris, residue, particulate, nano-particulate or the like from entering the inner lumen 24 of the tubular member 20. For example, some of the methods disclosed herein may utilize laser processing and/or chemical treatment to create a slot in the wall of a tubular member while minimizing the amount of debris, residue, particulate, nano-particulate or the like from entering the inner lumen 24 of the tubular member 20.

In at least some embodiments, slots 22 may be formed in tubular member using a laser cutting process. The laser cutting process may include essentially any suitable laser and/or laser cutting apparatus. For example, the laser cutting process may utilize a femtosecond laser. Other lasers or laser systems may also be used, as appropriate.

Utilizing processes like laser cutting may be desirable for a number of reasons. For example, laser cutting processes may allow tubular member 20 to be cut into a number of different cutting patterns in a precisely controlled manner. This may include variations in the slot width (which also may be termed “kerf”), ring width, beam height and/or width, etc. Furthermore, changes to the cutting pattern can be made without the need to replace the cutting instrument (e.g., a blade). This may also allow smaller tubes (e.g., having a smaller outer diameter) to be used to form tubular member 20 without being limited by a minimum cutting blade size. Consequently, tubular members 20 may be fabricated for use in neurological devices or other devices where a small size may be desired.

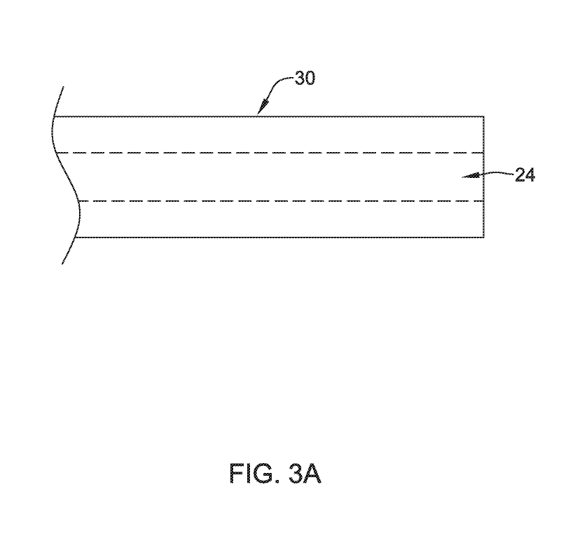

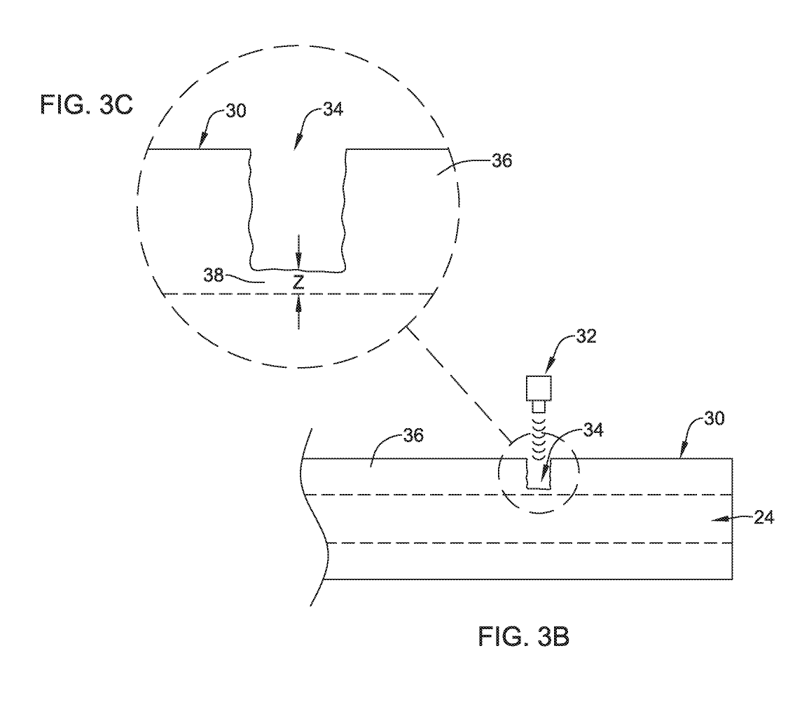

FIG. 3A shows an example “uncut” or “unslotted” tubular member 30. Tubular member 30 may be the “uncut” or “unslotted” version of tubular member 20. For example, FIG. 3A shows inner lumen 24 extending along the longitudinal axis of tubular member 30. A laser cutting process may generally include providing tubular member 30 and affixing tubular member 30 to a suitable holding apparatus. The holding apparatus may include one or more motors that can be used to rotate and/or translate tubular member 30 relative to the laser.

FIG. 3B shows example tubular member 30 positioned adjacent example laser 32. Further, FIG. 3B illustrates laser 32 applying laser energy to tubular member 30 to create cavity 34. As shown in FIG. 3B, application of laser energy may remove a portion of tubular wall 36. In some instances, laser energy applied to tubular wall 36 may ablate the tubular wall material.

FIG. 3C illustrates a detailed view of the cavity 34 created in tubular member 30. For the purposes of this disclosure, cavity 34 may be understood to be an opening or channel that is formed in tubular member 30 that extends only part way through the wall 36 of tubular member 30. A cavity 34 may alternatively be termed a pocket, trough, channel, groove, or the like. In some instances, laser 32 may form more than one cavity 34 in tubular member 30. Further, cavity 34 may have any of the configurations disclosed for slots 22 and/or any other suitable configuration.

As stated above, in some instances cavity 34 may extend only part way through the tubular wall 36 of tubular member 30. For example, FIG. 3C shows a portion of the tubular wall 36 removed to create cavity 34. As shown in FIG. 3C, the “thickness” of the remaining portion 38 of tubular wall 36 has been labeled “Z.”

As stated above, in some instances, tubular member 30 may be affixed to a holding apparatus in order to spin and/or translate tubular member 30 relative to laser 32. It can be appreciated that applying laser treatment (e.g. laser energy) to tubular member 30 while tubular member 30 spins or translates on a holding apparatus may remove a portion of the tubular wall along both an arc length (measured along the circumference of outer surface) and an axial width (measured parallel to the central axis of tubular member along outer surface) of the tubular member.

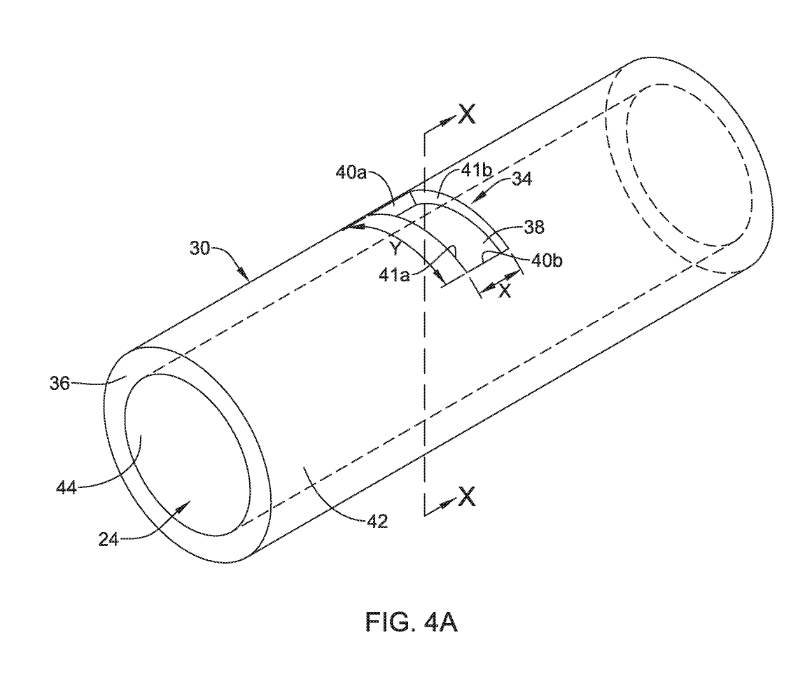

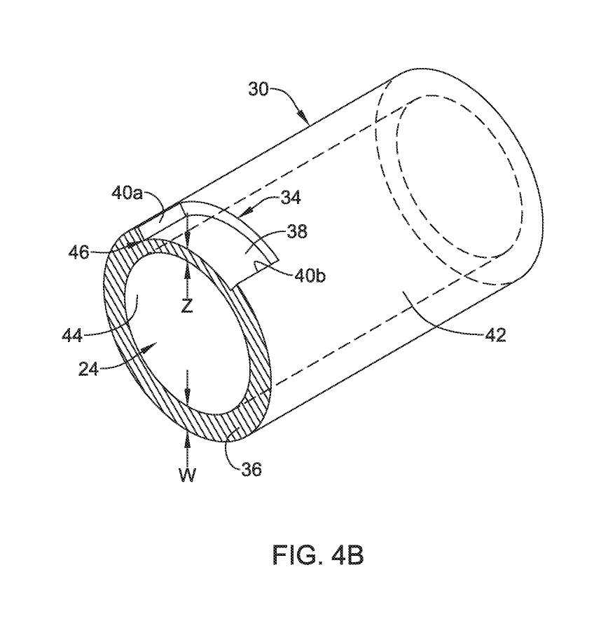

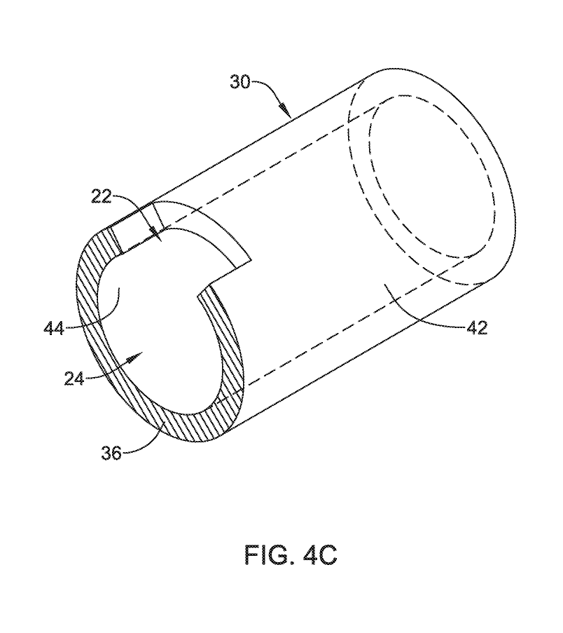

For example, FIG. 4A is a perspective view of example tubular member 30. As shown, a portion of the tubular wall 36 has been removed (by application of laser energy, for example) to form cavity 34. As discussed above, a portion of the tubular wall 36 has been removed along an arc length (measured along the circumference of outer surface 42) and an axial width (measured parallel to the central axis of tubular member 30 along outer surface 42) of tubular member 30. In FIG. 4A, the portion of the tubular wall 36 removed along the axial width of tubular member 30 may be represented by “X.” Axial width “X” may extend from a first axial width wall 41a longitudinally to a second axial width wall 41b. Further, the portion of the tubular wall 36 removed along the arc length of tubular member 30 may be represented by “Y.” Arc length “Y” may extend from a first arc length wall 40a to a second arc length wall 40b. Additionally, FIG. 4A depicts remaining portion 38 of tubular wall 36 after laser processing. In some instances, remaining portion 38 may alternatively be termed the “bottom” or “floor” of cavity 34.

FIG. 4B shows a cross-sectional view of cavity portion 34 (along line X-X of FIG. 4A). As described above, cavity portion 34 may include remaining portion 38 of tubular wall 36 extending between a first arc length wall 40a and a second arc length wall 40b. In some instances, remaining portion 38 extends continuously between first arc length wall 40a and second arc length wall 40b. In other words, in some instances there are no breaks, voids, apertures, or the like across the length and width of remaining portion 38. However, in other instances, remaining portion 38 may be discontinuous and include voids, breaks, apertures or the like distributed across its surface.

As shown in FIG. 4B, first and second wall portions 40a and 40b may define the thickness and/or depth of the amount of material which was removed from the outer surface 42 of tubular member 30 to create cavity 34. In other words, during laser processing, material may be removed from the outer surface 42 of tubular member 30 to a location 46 between the outer surface 42 and the inner surface 44 of tubular member 30.

As further illustrated in FIG. 4B, remaining portion 38 of tubular wall 36 may have a thickness represented by “Z.” Thickness “Z” may be defined as the distance from the location 46 to the inner surface 44 of tubular member 30.

In some instances, it may be desirable to precisely control the amount of material removed from the tubular wall 36 such that the thickness “Z” is minimized. For example, laser processing parameters may be controlled such that the thickness “Z” is approximately less than 1%, 5%, 10%, 15%, 20% or 50% of the unprocessed wall thickness “W” (shown in FIG. 4B) of tubular member 30. Further, in some instances the unprocessed wall thickness “W” may be approximately 0.0005 inches to 0.050 inches or more (e.g. 0.0010 inches to 0.025 inches or more, or about 0.001 inches to 0.007 inches or more).

In addition to forming cavity 34 in tubular member 30, tubular member 30 may also be subjected to additional method and/or processing steps. These steps may include a chemical treatment step. In some embodiments, it may be desirable to perform a chemical treatment step after a laser processing step.

Chemical treatment may include any number of processes including chemical etching. Chemical etching may include, for example, bathing tubular member 30 in an acid bath. The acid bath may include essentially any suitable acid. For example, the acid bath may include fluoroboric acid, nitric acid, any suitable bench and/or mineral acid, combinations thereof, or any other suitable acid. In some embodiments, the acid bath may include an aqueous solution including fluoroboric acid (e.g., about 1-20% or more, or about 2-10% or more, or about 4% or more) and nitric acid (e.g., about 1-50% or more, or about 20-40% or more, or about 30% or more). It can be appreciated that the various solutions that may be appropriate for the various bathes may vary depending on the material(s) used for tubular member 30.

Chemical treatment (e.g. chemical etching) may be desirable for a number of reasons. For example, after the laser cutting process is completed, tubular member 30 may have waste products or dross disposed along the interior of tubular member 30. The dross may comprise the parts or scraps of tubular member 30 that are removed from tubular member 30 in forming slots 22. The dross may take the form of a dust-like or bead-like material that tends to accumulate along the various surfaces (e.g. the inner surface 44) of tubular member 30. In some instances, it may be desirable to avoid and/or eliminate depositing dross on the inner surface 44 of tubular member 30.

In addition to preventing dross from accumulating along the various surfaces (e.g. the inner surface 44 of tubular member 30), chemical etching may also remove portions of tubular member 30. This may be desirable for a number of reasons. For example, by virtue of removing portions of tubular member 30, chemical etching may be used to create a variety of different slot configurations and/or orientations. This may include removing portions of tubular member 30 at or adjacent slots 22 via a chemical etching process.

Therefore, as stated above, it may be desirable to apply chemical treatment (e.g. chemical etching) to tubular member 30 after laser processing in order to complete the creation of slots 22. FIGS. 4B and 4C illustrate tubular member 30. In FIG. 4B, tubular member 30 is shown prior to a chemical etching step. Conversely, FIG. 4C illustrates tubular member 30 following a chemical etching step. At least some of the structural differences that may be incorporated into tubular member 30 using chemical etching can be seen by comparing FIG. 4B and FIG. 4C.

For example, FIG. 4C shows slot 22 as the open space created in the location where the remaining portion 38 (e.g. the “bottom” or “floor” of cavity 34 of tubular member 30) had been prior to chemical treatment. As stated above, it is contemplated that tubular member 30 (referred to above) may turn into tubular member 20 (shown in FIG. 2) after laser and/or chemical processing steps. In other words, after the removal of remaining portion 38 (e.g. by chemical treatment), cavity 34 may develop into slot 22 (shown in FIG. 4C). Therefore, FIG. 4C shows a cross-section of slot 22 having open space in the location where the remaining portion 38 had been prior to chemical treatment.

In some instances, chemical treatment may vary (e.g. remove material from) surfaces of tubular member 30 (e.g. the inner surface/diameter, the outer surface/diameter, the wall thickness, or both of tubular member 30). This may include removing portions of tubular member 30 along the outer diameter, inner diameter, or both via a chemical etching process. Consequently, in some instances the dimensions of tubular member 30 may change as material is removed from a particular portion of tubular member 30. For example, chemical treatment (e.g. chemical etching) may result in an increase in the inner diameter when material is removed from the inner surface, a decrease in the outer diameter when material is removed from the outer surface and/or a reduction in wall thickness as material is removed from both the inner and outer surfaces.

Furthermore, the chemical etching process may act on one or more surfaces of slots 22, thereby increasing the size of the slot as material is removed from the one or more slot 22 surfaces.

In some instances, chemical treatment of tubular member may uniformly remove material from all the surfaces of tubular member 30. However, in other instances material may be selectively removed from one or more surfaces of tubular member 30. For example, selected portions of tubular member 30 may be “masked” and thereby shielded from the chemical etching process. In some instances, material may be selectively removed from one or more surfaces of slots 22 to customize the slot 22 dimensions, configuration and/or shape.

For example, in some instances, chemical etching may be applied to tubular member 30 to the extent that remaining portion “Z” is removed and slot 22 is created. It can be appreciated that chemical etching may remove approximately one half of thickness “Z” from the “top” of remaining portion 38 and one half of thickness “Z” from the “bottom” (e.g. the inner surface of tubular member 30) of remaining portion 38. Furthermore, one half thickness “Z” may also be removed from the entire outer surface of tubular member 30 and one half thickness “Z” may also be removed from the entire inner surface of tubular member 30.

In at least some embodiments, chemical etching may occur while moving tubular member 30 in and out of an acid solution and/or rotating tubular member 30 within an acid solution. For example, tubular member 30 may be etched by bringing tubular member 30 into and out of a bath material, rotating tubular member 30 in a bath, or both. In some instances, however, rotation or translation may not be required. Therefore, fluid agitation or flow may be used instead. In other instances, no motion may be implemented. In some embodiments, the speed at which tubular member 30 is translated and/or rotated may vary. In general, the rate of motion may correlate to the rate of mass-removal from tubular member 30. It can be appreciated that the longer that tubular member 30 is in a given bath material, the greater amount of material that may be removed from tubular member 30. Thus, if one end of tubular member 30 spends more time in bath material, more material along that end may be removed (resulting in greater changes in inner diameter, outer diameter, slot width, or combinations thereof at that end). This property may be altered, for example, by masking portions of tubular member 30 so that one or more structural characteristic (e.g., inner diameter, outer diameter, slot width, etc.) may be left unaltered be altered to a lesser extent.

As stated, tubular member 20 (shown in FIG. 2) may represent tubular member is 30 after laser and/or chemical processing. Further, as shown in FIG. 2, slot 22 extends from outer surface 42 to inner surface 44 of tubular member 20. In other words, the combination of the laser processing and chemical treatment has removed an entire portion of tubular wall thickness 36 that defines slot 22. It is contemplated the methodology of utilizing laser processing in combination with chemical treatment may create more than one slot 22 in tubular member 20. As stated above, performing chemical treatment after laser processing may prevent and/or minimize the amount of dross and/or debris transferred to the inner surfaces of the tubular member 20. As stated above, the methodology may create a variety of slot combinations, sizes, groupings, shapes, geometries, or the like.

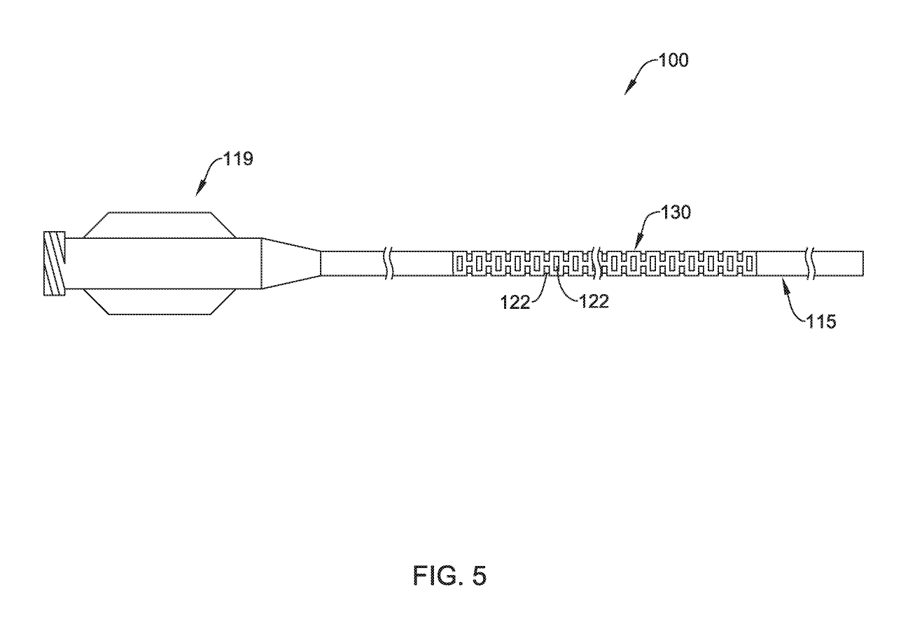

The forgoing discussion indicates that in at least some embodiments, tubular member 30 may be a part of guidewire 10. However, this is not intended to limit the scope of the invention as tubular member 30 may be used in essentially any other suitable medical device. For example, FIG. 5 illustrates another example medical device 100 that takes the form of a catheter and may include a tubular member 130, which may be similar in form and function to tubular member 30. For example, tubular member 130 may include a plurality of slots 122, which may be configured and/or arranged similarly to slots 22.

Further, in some instances a proximal portion of tubular member 130 may be coupled to a manifold 119 and a distal portion of tubular member 130 may be coupled to a distal tip 115. As described above, tubular member 130 may have one or more slots 122 extending through the tubular wall of tubular member 130. While slots 122 are shown as generally spaced equidistant and uniformly, it is contemplated that slots 122 may be arranged in a variety of configurations, distributions, orientations (both longitudinally and radially), arrangements, or the like. It should be understood that this disclosure is, in many respects, only illustrative.

Changes may be made in details, particularly in matters of shape, size, and arrangement of steps without exceeding the scope of the invention. The invention's scope is, of course, defined in the language in which the appended claims are expressed.

What is claimed is:

1. A method of manufacturing a medical device, the method comprising:
laser cutting a tubular member, the tubular member having an inner surface, an outer surface and a tubular wall defining a thickness extending therebetween, wherein laser cutting the member includes removing a portion of the thickness of the tubular wall at one or more discrete locations along the tubular member; and
chemically etching the one or more discrete locations to form a slot within the tubular wall at the one or more discrete locations along the tubular member.

2. The method of claim 1, wherein laser cutting includes laser cutting with a femtosecond laser.

3. The method of claim 1, wherein forming a slot within the tubular wall includes removing the tubular wall from the outside surface to the inner surface of the tubular member.

4. The method of claim 1, wherein forming a slot within the tubular wall includes removing a portion of the tubular wall from the outside surface to a location between the outside surface and the inner surface of the tubular member.

5. The method of claim 1, wherein laser cutting the tubular member includes ablating a portion of the tubular wall.

6. The method of claim 1, wherein laser cutting to remove a portion of the thickness of the tubular wall includes removing at least 80% of the thickness of the tubular wall.

7. The method of claim 1, wherein laser cutting creates a first wall portion and a second wall portion, and wherein the first wall portion is longitudinally aligned with the second wall portion and wherein a connecting portion extends between the first wall portion and the second wall portion.

8. The method of claim 7, wherein the connecting portion is continuous.

9. The method of claim 7, wherein the connecting portion is discontinuous.

10. The method of claim 1, further comprising performing laser cutting prior to chemical etching.

11. A method of manufacturing a medical device, the method comprising:
laser cutting a tubular member, the tubular member having an inner diameter, an outer diameter and a tubular wall defining a thickness, wherein laser cutting the member includes removing a portion of the thickness of the tubular wall to form one or more cavities in the tubular wall; and
chemically etching the tubular member to form a slot within the tubular wall at the one or more cavities along the tubular member.

12. The method of claim 11, wherein laser cutting includes using a femtosecond laser.

13. The method of claim 11, wherein removing a portion of the thickness of the tubular wall includes removing at least 80% of the tubular wall.

14. The method of claim 11, wherein laser cutting includes ablating a portion of the tubular wall.

15. The method of claim 11, wherein chemically etching the tubular member includes bathing the tubular member in an acid bath while rotating the tubular member, translating the tubular member, or both.

16. A medical device, comprising:
an elongate shaft including a tubular member, the tubular member having an inner surface, an outer surface, a tubular wall extending between the outer surface and the inner surface and a plurality of slots extending from the outer surface to the inner surface; and
wherein the plurality of slots are created by laser cutting one or more cavities in the tubular wall at one or more discrete locations along the outer surface of the tubular member and chemically etching the one or more cavities.

17. The medical device of claim 16, wherein laser cutting includes using a femtosecond laser.

18. The medical device of claim 16, wherein laser cutting includes ablation a portion of the tubular wall.

19. The medical device of claim 16, wherein creating the one or more cavities includes removing a portion of the tubular wall, and wherein chemically etching includes removing the remaining portion of the tubular wall.

20. The medical device of claim 19, wherein removing a portion of the tubular wall includes removing at least 80% of a thickness of the tubular wall.
